(19) United States
(12) Patent Application Publication
(10) Pub. No.: US 2009/0197231 A1
(43) Pub. Date: Aug. 6, 2009

(54) Toilet training using absorbent article packaging

(76) Inventor: Paula Mary Sosalla, Appleton, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 12/012,936

(22) Filed: Feb. 6, 2008

(51) Int. Cl. G09B 23/00; A61B 17/00; B65D 73/00; B65D 85/00

(52) U.S. Cl. 434/267; 434/262; 206/440; 206/457; 206/459.5

(57) ABSTRACT

Presented is packaging for supplying disposable personal care products to a caregiver and for enhancing toilet training of a child, the packaging including a storage bin having a storage bin bottom and a reclosable cover and defining an interior space; a plurality of disposable personal care products disposed in the interior space; and an informational item adapted to teach the caregiver to use the storage bin to enhance toilet training. Also presented is a method for toilet training a subject, the method including providing a storage bin having a storage bin bottom and a reclosable cover and defining an interior space, wherein a plurality of disposable personal care products disposed in the interior space; and instructing a caregiver to incorporate use of the storage bin into toilet training.

### BACKGROUND OF THE INVENTION

Absorbent articles such as diapers, training pants, incontinence products, feminine hygiene products, swim undergarments, and the like conventionally include a liquid permeable body-side liner, a liquid impermeable outer cover, and an absorbent core. The absorbent core is typically located in between the outer cover and the liner for taking in and retaining liquids (e.g., urine) exuded by the wearer.

Personal care products such as toiletries, moist and dry wipes, diapers, training pants, incontinence products, feminine hygiene products, swim undergarments, and the like, are conventionally packaged in discardable packaging, including significant portions that are not or cannot be recycled. Currently, personal care items are normally packaged in a poly bag or box that is used for shipping and retail sale and then thrown away.

At the same time, toilet training is an important stage in a child's growth, and many absorbent articles have been adapted for use in a training program, such as toilet training or enuresis control. Various types of sensors and indicators, including moisture or wetness indicators, have been suggested for use in absorbent articles. Problems, however, have been encountered in using such articles for training and/or notification purposes in that such training aids can be expensive and complicated.

### SUMMARY OF THE INVENTION

As caregivers undertake programs to address toilet training, enuresis control, or incontinence, which apply across demographics, those caregivers would benefit by gaining greater access to products and guidance that are specifically designed to enhance the effectiveness of such programs and to solve waste problems associated with a lack of reuse of packaging.

The invention described herein solves the problems described above and provides an increase in toilet training efficacy using disposable personal care articles and their packaging. The invention described herein is a unique method of combining disposable personal care articles and their packaging into a training regimen.

The present inventors undertook intensive research and development efforts concerning improving the delivery of personal care products, particularly with respect to providing packaging that can be recycled and/or reused. The packaging system not only serves to contain and identify personal care products for shipping and retail purposes, but also serves to assist with toilet training as described below.

### BRIEF DESCRIPTION OF THE DRAWINGS

The foregoing and other features and aspects of the present invention and the manner of attaining them will become more apparent, and the invention itself will be better understood by reference to the following description, appended claims and accompanying drawings, where:

FIG. 1 is a partially-cutaway perspective view of the packaging system of the present application, where the packaging system includes a storage bin with a cover.

Repeat use of reference characters in the present specification and drawings is intended to represent the same or analogous features or elements of the present invention. The drawings are representational and are not necessarily drawn to scale. Certain proportions thereof may be exaggerated, while others may be minimized.

### DETAILED DESCRIPTION OF THE INVENTION

It is to be understood by one of ordinary skill in the art that the present discussion is a description of exemplary aspects of the present invention only, and is not intended as limiting the broader aspects of the present invention.

The present disclosure is generally directed to personal care products and how they are packaged for transport, retail sale, and home use. The personal care product can be, for instance, a moist or dry wipe, a toiletry, or an absorbent article such as a diaper, a training pant, an incontinence product, a feminine hygiene product, a medical garment, a bandage, or the like. Generally, the personal care products are disposable, meaning that they are designed to be discarded after a limited use rather than being laundered or otherwise restored for reuse.

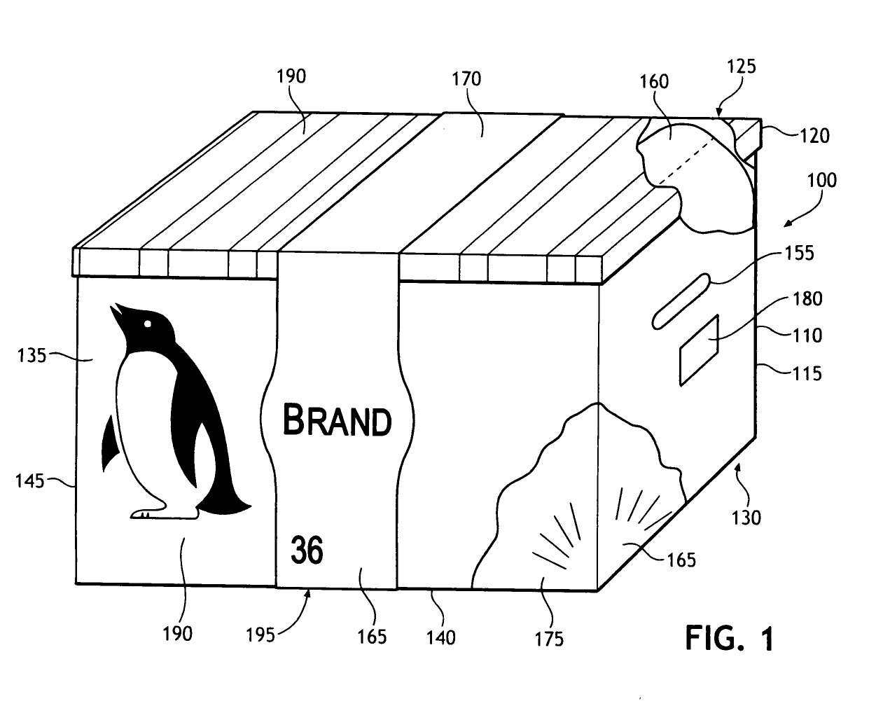

The packaging described herein is particularly useful for transporting and selling disposable personal care products including absorbent articles, wipes, toiletries, and other personal health and hygiene items. Referring to FIG. 1 for exemplary purposes, a packaging system 100 is shown. The packaging system 100 is adapted such that all or most of the packaging system 100 is readily reused and/or recycled.

As illustrated in FIG. 1, the packaging system 100 includes a storage bin 110 as its central component. The storage bin 110 includes a storage bin bottom 115 and a cover 120. The cover 120 can be formed integrally with the storage bin bottom 115. In other aspects, the cover 120 can be either partially or completely removable from the storage bin bottom 115, and can be capable of being reattached to or re-set upon the storage bin bottom 115 to reclose the storage bin 110. The cover 120 can be a separate piece that is attached to the storage bin 110 with a tab-and-slot mechanism that forms a usable, flip-top opening and closing lid. In another aspect, a flip-top cover can be a component of the storage bin bottom 115 and can form a usable, flip-top opening and closing lid employing a scored, folded hinge. In other aspects, the cover 120 can be in the form of single or multiple flaps. The storage bin bottom 115 and cover 120 together define an interior space 125 of the storage bin 110.

The interior space 125 can include dividers or other structure suitable for breaking up the interior space 125 into smaller portions. The cover 120 of the storage bin 110 can also be configured such that it is a drawer that is slidably inserted into the interior space 125 of the storage bin 110. Positioning the storage bin 110 such that the storage bin opening is on a side of the storage bin 110 enhances the use of the drawer and the appearance of the storage bin 110 as a dresser. In this aspect, the drawer can include dividers or other structure suitable for breaking up the drawer into smaller portions.

The storage bin 110 as illustrated is generally rectilinear, but can be any suitable shape. The storage bin bottom 115 includes a lower face 130 and, if rectilinear, also includes four sides. If, for example, the storage bin 110 is generally cylindrical, the storage bin bottom 115 will include only one side 135 extending around the perimeter of the lower face 130. The storage bin 110 has a footprint, which is generally the view of the lower face 130 either from below or from the interior space 125. If the storage bin 110 is rectilinear, then the footprint will be rectilinear as well. If the storage bin 110 is cylindrical, then the footprint will be circular. The footprint can be any other suitable shape including an oval, a hemisphere, a triangle, a square, or any other shape including linear and/or arcuate portions.

The storage bin 110 can be manufactured from any suitable material including corrugated cardboard, injection-molded or other plastic, wood, woven or nonwoven fabrics, or combinations of these. As a result, the storage bin 110 can be rigid or flexible, or a combination of these. Part or all of the storage bin 110 can be opaque, translucent, or generally transparent, and can be of any suitable color. In one example, the storage bin 110 can be manufactured from corrugate sturdy enough to support reuse of the storage bin 110 by the consumer. In another example, the storage bin 110 can be manufactured from multi-layered, insulated, liquid-resistant fabric such that a consumer can reuse the storage bin 110 as a cooler. In still another example, the storage bin 110 can be manufactured from plastic such that the consumer can reuse the storage bin 110 for semi-permanent storage, as a planter, or for any other typical household use.

Packaging used prior to this invention is optimized to minimize material usage; it is designed to be only durable enough to get products to a consumer. As a result, current packaging is not durable and has limited reuse possibilities. Packaging of the present disclosure is made to be more durable and thus have uses beyond shipping and retailing purposes.

There are many storage bin styles including but not limited to, slotted boxes, telescoping boxes, folders, rigid boxes such as Bliss boxes, self-erecting boxes, corrugated common footprint containers, and interior forms. Interior forms such as liners, tubes, pads, build-ups, dividers, partitions, and other inner packaging pieces can be made in an infinite variety of ways to separate or cushion products, to strengthen the storage bin 110, or to fill voids to prevent products from moving.

Also, in the aspects of the present disclosure in which the storage bin 110 is made from corrugate, corrugated fiberboard, or combined board, has two main components, the linerboard and the medium. Both are made of a special kind of heavy paper called container board. Linerboard is the flat facing or liner that adheres to the medium. The medium is the corrugated or fluted paper glued between the linerboard facings. Linerboard can be single face, single wall, double wall, or triple wall. Flutes come in several common sizes or profiles ranging from A (33 flutes/foot) to F (125 flutes/foot). Smaller flute profiles provide enhanced structural and graphics capability for primary and retail packaging, while larger flute profiles deliver a greater vertical compression strength and cushioning. Different flute profiles can be combined in one piece of combined board. Mixing flute profiles allows designers to manipulate the compression strength and total thickness of the combined board.

Corrugating adhesive is normally bonded to the liners with a starch-based adhesive. It is available in several levels of water resistance. A flat piece of corrugated fiberboard that has been cut, slotted, and scored is called a box blank. For some box styles, in order to make a box, the two ends of the box blank must be fastened together with glue, tape, or staples. The place where these two ends meet is known as the manufacturer's joint. For storage bins 110 of the present disclosure, a glue joint on the inside of the storage bin 110 is preferred. This is done for both aesthetics and safety reasons. Also, for the storage bin 110 and cover 120 in general, it is desired to have the entire structure glued versus taped or stapled.

Accurate inside storage bin dimensions must be determined to ensure the proper fit of the products being shipped or stored. In addition, accurate outside storage bin dimensions must be determined to optimize palletizing and distributing the product. For storage bins 110 of the present disclosure, storage bin dimensions are influenced by several factors including: consumer-preferred storage bin dimensions, retailer-desired price point of offering, product quantity, product size and configuration (e.g., arrangement and orientation), pallet footprint and cube utilization, and manufacturing equipment limitations. Storage bin sizes and dimensions can also be reflective of dresser-drawer-type dimensions. For example, the storage bin 110 can be of a rectangular structure having a greater width than height.

In other aspects of the present disclosure, windows or special cut outs can be added to the storage bin 110 to enhance functionality or aesthetic appeal, including to add air vents to the storage bin 110. In still other aspects of the present disclosure, the storage bin 110 can include a second storage bin 110 inside for strength.

The cover 120 can include a latching element (not shown) to allow the cover 120 to be positively attached to the storage bin bottom 115. The latching element can be any suitable mechanism, including adhesives, rigid or flexible tabs that engage a slot or other device, elements to allow the cover 120 to be snapped onto the storage bin bottom 115, a simple friction fit, hardware such as hinges and hasps, or any combination of these. The cover 120 can have an indentation sized to the outline of the lower face 130 of another storage bin 110 to assist in positioning and to provide stability while stacking storage bins 110.

The storage bin 110, as it is presented to the consumer, includes disposable personal care products within the interior space 125 of the storage bin 110. Such disposable personal care products include, but are not limited to, absorbent articles, dry, moist, or wet wipes, toiletries, and other personal care items. Absorbent articles include diapers, training pants, swim pants, incontinence products, feminine hygiene products, bandages, and the like. The personal care products can be disposed within the interior space 125 in any suitable arrangement.

In another aspect of the present disclosure, the personal care products can be disposed within one or more inner packages 160. Such inner packages 160 can provide compression, containment, environmental protection, and/or organization to the personal care products. In one aspect, the inner package 160 is a flexible consumer package, which refers to non-rigid containers, such as polyethylene bags, that are adapted to contain personal care products and are adapted to be presented to a consumer. In other various aspects, the inner package 160 is manufactured from standard poly film, paper, woven or nonwoven fabric, or from any other suitable material or combination of materials and in any suitable manner.

Product-identifying graphics 195 can include the type of personal care product disposed in the interior space 125, the size or sizes of personal care products disposed in the interior space 125, and the count, amount, or volume of personal care products disposed in the interior space 125, all as applicable to the type of personal care products. Product-identifying graphics 195 can also include the manufacturer, customer service information, use instructions, patent markings, and any other information typically disposed on a package of personal care products.

Further, to enhance a consumer's and a child's interest in reusing the storage bin 110, the storage bin 110 includes non-product-identifying graphics 190. The non-product-identifying graphics 190 can include designs, patterns, characters, or any other suitable graphical elements, including those that might be attractive to a child or other user of the storage bin 110. For example, the non-product-identifying graphics 190 can include depictions of a popular cartoon character as well as depictions of items and/or colors associated with that character. The non-product-identifying graphics 190 on the storage bin 110 can be registered in relation to the storage bin bottom 115. The non-product-identifying graphics 190 on the storage bin 110 can include graphical elements such as labels, hinges, hasps, locks, and other elements to enhance the look of the storage bin 110. The non-product-identifying graphics 190 can further include textured elements such as those that can be supplied by embossing. In another aspect, non-product-identifying graphics 190 can be applied or otherwise disposed within the interior space 125 of the storage bin 110. The graphics can include a label area 180 to allow the consumer to label the storage bin 110 with a child's name or other desired or identifying information. The label area 180 can be positioned on any face or surface of the storage bin 110.

Still further, the non-product-identifying graphics 190 can include graphical elements designed to make the storage bin 110 resemble a household item, including a household storage item such as a chest, a dresser, a cabinet, a toy box, a toy, a wardrobe, a trunk, a jewelry box, or a similar item. For example, the addition of real or graphical hinges and pulls and coordinating graphics can make the storage bin 110 resemble such a household storage item. In other aspects of the present application, the storage bin 110 can include one or more attachments or accessories to enhance the resemblance of the storage bin to a household item, including simulated feet, handles, or backs. Such attachments or accessories can be attached to the storage bin 110 or included within the storage bin 110 by the manufacturer, or may be provided to the consumer by any suitable means for further assembly by the consumer.

The storage bin 110 can include a label or other space in which the child's name can be written, helping to make the drawer the child's special possession. In the aspect in which the storage bin 110 includes at least one drawer, multiple storage bins can be stacked to form a dresser in which to keep other articles of clothing, small toys, etc.

The packaging system 100 can also include a bin enclosure 165 that partially or completely envelops the storage bin 110. The bin enclosure 165 can assist in ensuring the cover 120 stays in place atop the storage bin bottom 115 through manufacturing, shipping, and retail handling. The bin enclosure 165 can also provide space on which to provide product, brand, and other labeling information. The bin enclosure 165 can also provide environmental protection to shield the storage bin 110 from dust, dirt, water, and other hazards that might otherwise adversely affect the appearance, performance, and value of the storage bin 110 and the products within.

In one aspect of the present disclosure, the bin enclosure 165 is a sleeve 170 that surrounds all or part of the vertical perimeter 145 of the storage bin 110. In general, the sleeve 170 encircles the storage bin 110 to provide both a means of keeping the cover 120 in place atop the storage bin bottom 115 and an additional surface on which to provide product, brand, and other labeling information. The sleeve 170 has a bin-facing side and an outward-facing side. The sleeve 170 can be manufactured from paperboard, poly film, woven or nonwoven fabric, or any other suitable material or combination of materials. The sleeve 170 is wrapped around the storage bin 110 and then attached to itself and/or the storage bin 110 using adhesives, tabs, or any other suitable mechanisms. The sleeve 170 can be centered on the storage bin 110 or can be offset toward one side of the storage bin 110. In other aspects of the present disclosure, windows or special cut outs can be added to the sleeve 170 to enhance functionality or aesthetic appeal.

In another aspect of the present disclosure, the bin enclosure 165 is an overwrap 175 that surrounds all or part of the storage bin 110. In general, the overwrap 175 encloses the storage bin 110 to provide a means of keeping the cover 120 in place atop the storage bin bottom 115; an additional surface on which to provide product, brand, and other labeling information; and environmental protection of the storage bin 110 and its contents. The overwrap 175 can be manufactured from poly film, polarizing film, woven or nonwoven fabric, or any other suitable material or combination of materials. The overwrap 175 is wrapped around the storage bin 110 and then attached to itself and/or the storage bin 110 using heat, shrinking, adhesives, tabs, or any other suitable mechanisms.

In general, the bin enclosure 165, whether it is a sleeve 170 or an overwrap 175, can include indicia indicating a secondary use of the bin enclosure 165. In addition, the packaging system 100 can include both a sleeve 170 and an overwrap 175. In this aspect, the sleeve 170 is typically placed directly on the storage bin 110 as a means of keeping the cover 120 in place atop the storage bin bottom 115. The overwrap 175 is then placed to enclose both the storage bin 110 and the sleeve 170 as environmental protection of the storage bin 110 and its contents. In this aspect, either or both of the sleeve 170 and the overwrap 175 can be used as a surface on which to provide product-identifying graphics 195 including product, brand, and other labeling information.

The storage bin 110 can also include at least one hand hold 155 adapted to accommodate at least a portion of a human hand to facilitate grasping the storage bin 110. The hand hold 155 can be positioned on any side, on the lower face 130, or on the cover 120. The storage bin 110 can alternatively include a plurality of hand holds 155 positioned on any or all of those locations. The hand hold 155 can be a hole in the storage bin 110. The hand hold 155 can also be a handle or any other suitable device adapted to accomplish the purpose of grasping the storage bin 110.

The features selected for the storage bin 110 are selected to be representative of the reuse of the storage bin 110. Such features include the hand holds 155 and the cover 120, along with other features described herein. Such features ensure that the storage bin 110 functions properly during reuse.

A unique aspect of the storage bin 110 described herein is that it is also a training aid to be used in the toilet training process. Children are aware that their clothing is kept in dresser drawers, in a closet, etc. Choosing what they want to wear and trying to dress themselves is one of the first steps to becoming a big kid and it is a step that children find fun and exciting. At the same time, choosing clothing is a real step towards autonomy for them. Keeping the child's absorbent articles designed for training in the child's own special place, such as a drawer, puts more focus on the product as more underwear-like, and less like a diaper, because diapers are for babies and are kept in a bag or in a stack on the changing table.

This practice of keeping absorbent articles in the child's own drawer, for example, meets many of the criteria for a successful training aid. It is motivational for the child. It teaches that absorbent articles designed for training are more like clothing/underwear than like a diaper. It makes the child feel special because the drawer is their possession. It makes the child feel grown-up because they can open and close the storage themselves, and can even have their name on it. It can even precipitate the move into real, cloth underwear by eventually keeping some cloth underwear in “their drawer” in addition to their training absorbent articles to wear during the day or whenever they feel like trying real underwear.

Typical packaging used to contain and store absorbent articles does not add additional value beyond containing and storing the articles. In some cases, a corrugate container may have another use as a storage container. The present application has additional value beyond simple containment and storage of absorbent articles; the packaging system 100 of the present application is itself a toilet training aid in the toilet training process. The packaging system 100 of the present application is structured to appear as a dresser or chest where normal undergarments and other apparel are stored. The present application helps teach awareness that the absorbent articles being stored inside, such as training pants, can be considered like underwear and are a step in the toilet training process.

As noted above, it is the special features, both structural and graphical, that aid the appearance of a household storage item such as a dresser or storage chest. Structural features can include a flip-top lid, which is easy for a child to open by themselves, feet at the bottom four corners of the storage bin 110, a pull-out drawer, or handles or knobs for opening. Graphical design features include graphics that look like a drawer, dresser feet, handles or drawer pulls, and the like. Another graphical design feature that teaches toilet training is a progress chart or other type of reward chart.

The special features designed to make the package easy for a child to use set this application apart from typical packaging. It is possible for a child to dispense training pants from a typical poly bag or corrugate box, but neither of those packages has the training advantages of the present application.

To enhance this approach, the packaging system 100 can also include an informational item meant to instruct a caregiver with respect to the approaches and benefits described above. The informational item can describe the philosophy behind treating the storage bin 110 as the child's own possession and in instructing the child to take ownership of the storage bin 110 and the absorbent articles within it. The informational item can also include information with respect to other tips and approaches to successful toilet training, including information regarding an appropriate response to an insult incident. Appropriate responses to an insult incident can include discussing physical sensations with the subject; informing the subject with respect to the relation between the physical sensations and the imminence of the insult; taking the subject to a bathroom; positioning the subject on a toilet; encouraging the subject to produce an insult; recognizing positive actions by the subject; and recognizing toilet training progress by the subject.

The informational item can also include information regarding tracking and assessing toilet training progress. In other aspects of the present disclosure, the informational item can include a growth chart, sizing information for shoes, sizing information for clothes, sizing information for absorbent articles, and/or other information useful to a caregiver or a child, printed or otherwise disposed thereon.

In various aspects, the packaging system 100 can include other informational items such as instructions in the use of the product and tips for toilet training, enuresis control, or incontinence control. As used herein, the term “informational item” refers to objects that are provided in addition to disposable personal care articles, are adapted to communicate information to the user and/or consumer of the disposable personal care articles, and are associated with individual components of the packaging system 100. Examples of informational items include cards, paper, electronic media, printing on the packaging, or other suitable media capable of storing and conveying information. In another aspect of the present application, an informational item can be incorporated into the non-product-identifying graphics 190. For example, if the non-product-identifying graphics 190 include a car, a progress chart in the form of a racetrack can be incorporated into the non-product-identifying graphics 190.

In various aspects, the informational items associated with the packaging system components may be adapted to appeal to the specific category of user and/or purchaser to which the disposable personal care article is adapted. The informational items may be adapted, for example, by providing information likely to be of interest to a given category of user and/or purchaser.

For example, a disposable personal care article may be adapted for use by a caregiver for toilet training purposes. An informational item may be associated with the disposable personal care article that is adapted to interest caregivers. For example, the informational item may be a card containing information or instructions about children's health and hygiene, such as sleep habits, thumb sucking, teething, skin health, toilet training; questions to ask a child; jokes; and the like, and combinations thereof. The informational item may additionally or alternatively include addresses for web sites available on the Internet. The web sites may contain information related to issues of interest for caregivers and users of disposable personal care articles.

The informational item may additionally or alternatively include addresses for web sites available on the Internet or telephone numbers for audiovisual recorded information that can be played back by a computer or via telephone or other communication technology. The informational item may additionally or alternatively include information describing activities that are suitable for caregivers and users of disposable personal care articles. The activities may be adapted for a child at a specific age, size, and/or stage of development. For example, the activities may be adapted to promote interaction between the child and the caregiver.

Toilet training is described as an example of the use of these tools, but these tools are equally applicable to other situations such as incontinence training, enuresis training, and pre-toilet-training stages as well.

As described above, the caregiver is provided with instructions, guidance, and information with respect to the actions to follow to best use an insult as an opportunity to toilet train a subject. The caregiver is instructed to respond to an insult while the insult is occurring or as soon as possible thereafter. The caregiver and subject may respond by discussing the physical sensations sensed by the subject before the insult occurs and while the insult is occurring, such physical sensations to include sensations of pressure, temperature and pressure changes, wetness, and any other appropriate sensations. The caregiver and subject may also respond by discussing the relation between the physical sensations and the imminence of the insult. For example, the caregiver may point out the sensations experienced by the subject just prior to the insult and help the subject to associate those sensations with the imminence of an insult.

The caregiver and subject may also respond by making sure the subject is taken to a bathroom and by discussing the relationship between the insult and the bathroom. The caregiver and subject may also respond by making sure the subject is positioned on a toilet or other appropriate receptacle and by discussing the relationship between the insult and the toilet or other receptacle. The caregiver may also respond by encouraging the subject to produce an insult while positioned on the toilet or other receptacle, again discussing the relationship between the insult and the toilet or other receptacle. The caregiver may also respond by recognizing any positive actions taken by the subject to encourage the subject to perform additional positive acts.

Toilet training may be further enhanced by giving the caregiver instructions and tools with respect to tracking and assessing the toilet training progress of the subject. Understanding the progress or lack of progress being made by the subject allows the caregiver to adjust the toilet training to improve progress, as described herein.

These and other modifications and variations to the present invention may be practiced by those of ordinary skill in the art, without departing from the spirit and scope of the present invention, which is more particularly set forth in the appended claims. In addition, it should be understood that aspects of the various aspects of the present invention may be interchanged either in whole or in part. Furthermore, those of ordinary skill in the art will appreciate that the foregoing description is by way of example only, and is not intended to limit the invention so further described in such appended claims.

What is claimed:

1. Packaging for supplying disposable personal care products to a caregiver and for enhancing toilet training of a child, the packaging comprising:
a storage bin having a storage bin bottom and a reclosable cover and defining an interior space;
a plurality of disposable personal care products disposed in the interior space; and
an informational item adapted to teach the caregiver to use the storage bin to enhance toilet training.

2. The packaging of claim 1, wherein the storage bin is configured to resemble a household storage item.

3. The packaging of claim 2, wherein the storage bin is configured to resemble a dresser.

4. The packaging of claim 2, wherein the storage bin is configured to resemble a toy box.

5. The packaging of claim 2, wherein the storage bin is configured structurally to resemble a household storage item.

6. The packaging of claim 2, wherein the storage bin is configured graphically to resemble a household storage item.

7. The packaging of claim 1, wherein the storage bin includes dividers for dividing the interior space.

8. The packaging of claim 1, wherein the reclosable cover includes a drawer.

9. The packaging of claim 1, wherein the storage bin includes an attachment to enhance the resemblance of the storage bin to a household storage item.

10. The packaging of claim 1, wherein the storage bin includes child-focused graphics.

11. The packaging of claim 1, further comprising a removable bin enclosure adapted to overlie a portion of the storage bin and to be removable from the storage bin by a consumer, wherein the bin enclosure includes product-identifying graphics, and wherein the storage bin includes non-product-identifying graphics and is substantially free of product-identifying graphics.

12. The packaging of claim 1, wherein the storage bin includes non-product-identifying graphics, and wherein the non-product-identifying graphics include an informational item.

13. The packaging of claim 1, wherein the personal care products are training pants.

14. The packaging of claim 1, wherein the informational item includes information regarding an appropriate response to an insult incident.

15. The packaging of claim 1, wherein the informational item includes information regarding tracking and assessing toilet training progress.

16. A method for toilet training a subject, the method comprising:
providing a storage bin having a storage bin bottom and a reclosable cover and defining an interior space, wherein a plurality of disposable personal care products disposed in the interior space; and
instructing a caregiver to incorporate use of the storage bin into toilet training.

17. The method of claim 16, wherein instructing includes teaching the subject to consider the storage bin as being owned by the subject.

18. The method of claim 16, wherein instructing includes teaching the subject to obtain a personal care product from the storage bin.

19. The method of claim 16, wherein instructing includes teaching the subject to consider a personal care product to be a clothing item.

20. A packaging system for use as a toilet training aid, the packaging system comprising:
a storage bin configured to resemble a household storage item;
a removable bin enclosure adapted to overlie a portion of the storage bin and to be removable from the storage bin by a consumer; and
an informational item adapted to teach a caregiver to use the storage bin to enhance toilet training.
